(19) United States
(12) Patent Application Publication
(10) Pub. No.: US 2015/0252412 A1
(43) Pub. Date: Sep. 10, 2015

(54) HIGH-DEFINITION DNA IN SITU HYBRIDIZATION (HD-FISH) COMPOSITIONS AND METHODS

(71) Applicant: Massachusetts Institute of Technology, Cambridge, MA (US)

(72) Inventors: Alexander Van Oudenaarden, CT Utrecht (NL); Nicola Crosetto, Solna (SE); Marzena Magda Bienko, Solna (SE); Leonid Teytelman, Cambridge, MA (US)

(73) Assignee: Massachusetts Institute of Technology, Cambridge, MA (US)

(21) Appl. No.: 14/424,766

(22) PCT Filed: Aug. 30, 2013
(86) PCT No.: PCT/US13/57722

§ 371 Date: Feb. 27, 2015

Related U.S. Application Data
(60) Provisional application No. 61/696,096, filed on Aug. 31, 2012.

(51) Int. Cl. C12Q 1/68

(52) U.S. Cl. CPC C12Q 1/6841

(57) ABSTRACT

The invention provides methods and compositions relating to high definition fluorescence DNA in situ hybridization (HD-FISH). The probes generated using the methods of the invention demonstrate higher resolution and efficacy than conventional DNA FISH probes.

### RELATED APPLICATIONS

This application claims the benefit of U.S. Provisional Application No. 61/696,096, filed Aug. 31, 2012, the entire contents of which are incorporated by reference herein.

### FEDERALLY SPONSORED RESEARCH

This invention was made with government support under Grant No. DP1 OD003936 awarded by the National Institutes of Health. The government has certain rights in this invention.

### BACKGROUND OF INVENTION

The tremendous advancement of DNA fluorescent in situ hybridization (FISH) techniques during the past twenty years has revolutionized multiple areas of biomedicine, including prenatal screening¹ and onco-hematological diagnostics². In biology, the combination of DNA FISH with fixation procedures capable of maintaining the three-dimensional architecture of the nucleus has paved the way for studies exploring the spatial organization and dynamics of chromatin³⁻⁵. Nonetheless, DNA FISH remains limited by several important factors, including poor resolution (typically, probes cover regions of several hundred kilobases (kb), yielding patchy signals), low number and high cost of commercially available probes, as well as high cost and time required to synthesize custom-made probes. Finally, the signals produced by such probes are usually poorly amenable to automated image processing.

### SUMMARY OF INVENTION

The invention is aimed at improving resolution and processing of DNA FISH signals. Some aspects of the invention provide streamlined and cost-effective approaches to the preparation of probes. The invention allows an end-user to fully exploit the enormous potential of DNA FISH for both medical diagnostics and research.

In accordance with some aspects of the invention, the inventors have compiled, inter alia, a genome-wide database of optimally designed primer pairs for fast and cost-effective PCR-based synthesis of highly specific, single- or double-stranded DNA FISH probes, targeting each gene in the human genome plus thousands of inter-genic regions. These probes allow the visualization of as little as 3 kb, producing signals that are robustly quantifiable by image-processing software. The invention represents an improvement over existing DNA FISH methods for one or more of the following reasons: 1) the synthesis of probes is highly streamlined and cost-effective; 2) the resolution achieved using these probes is higher than typically achievable with commercially available probes; 3) the signal produced is amenable to robust and automated computational processing in a large number of cells; and 4) strand-specific probes can be produced. The probes identified, and optionally generated, using the methods of the invention can be used in a broad range of applications, including medical diagnostics, chromatin architecture studies, and analysis of combed DNA.

Thus, in one aspect, the invention provides a method comprising (a) identifying a plurality of nucleic acids, each having a nucleotide sequence that is unique relative to sequenced genomic DNA of a species; (b) identifying for each of the unique nucleic acids identified in (a) the following: (i) amplification primer pairs that generate nucleic acid fragments that are about 70 to about 300 base pairs (bp) in length, and (ii) the nucleic acid fragments that are generated through amplification with such identified primer pairs, thereby creating a putative primer set and a putative probe set; (c) eliminating (or excluding), from the putative probe set, nucleic acid fragments having amplification primer pairs that amplify more than one nucleic acid fragment or that amplify a single incorrect nucleic acid fragment from a nucleic acid sample (and accordingly optionally eliminating, from the putative primer set, primer pairs that amplify more than one nucleic acid fragment or that amplify a single incorrect fragment from the nucleic acid sample); and (d) eliminating (or excluding), from the putative probe set, nucleic acid fragments that hybridize to multiple regions in the genomic DNA of the species or that do not hybridize faithfully to their own region (and accordingly optionally eliminating, from the putative primer set, primer pairs that amplify such eliminated fragments. The method therefore yields either or both a fluorescence in situ hybridization (FISH) probe set and a FISH primer set. The probe set may be referred to herein as a high-definition FISH (HD-FISH) probe set, intending that at a minimum it can provide a high degree of resolution when used in FISH. Similarly, the primer set may be referred to herein as a HD-FISH primer set. As used herein, a primer set may be referred to as a probe primer set.

In some embodiments, step (c) is performed before step (d). In some embodiments, step (d) is performed before step (c).

In another aspect, the invention provides a method that comprises step (a) followed by step (d). In this aspect, the probes may have a length of 30-300 base pairs (or bases, if single stranded).

In some embodiments of these aspects, step (a) of the method is performed by analyzing a plurality of overlapping nucleic acid sequences along a contiguous genomic region such as a chromosome. In some embodiments, each of the overlapping nucleic acid sequences is about 500 bases in length. The extent of overlap between neighboring sequences may be about 400 bases.

In some embodiments of these aspects, the species is human.

One or more of the steps of the methods may be performed and/or the determinations associated with these steps may be made using in silico techniques (e.g., computer algorithms or simulations). In some embodiments, step (a) is performed in silico. In some embodiments, step (b) is performed in silico. In some embodiments, step (c) is performed in silico. In some embodiments, step (d) is performed in silico. In some embodiments, steps (a) through (d) are performed in silico. Computer programs that may be used include BLAT (e.g., for steps (a) and (d), Primer3 for step (b), and e-PCR for step (c), all of which are publicly available.

In some embodiments, step (a) excludes nucleic acids that have 80% or higher similarity to other sequences in the genome of a species. In some embodiments, step (d) excludes nucleic acids that have 70% or higher similarity to other sequences in the genome of a species. In this manner, step (d) can be thought of as being more stringent than step (a).

In some embodiments, the set of nucleic acid fragments identified in step (b), and therefore also the ultimate HD-FISH probe set, comprises fragments or probes that are about 30-300 bases in length. In some embodiments, the set of nucleic acid fragments identified comprises fragments or probes that are 70-300 bases in length. In some embodiments, either set is comprised of fragments or probes that are similar in length (e.g., +/−10 bases in length). In some embodiments, the fragments or probes may have an average length of about 30 bases (e.g., 30+/−5 bases), about 35 bases (e.g., 35+/−5 bases), about 40 bases (e.g., 40+/−10 bases), about 50 bases (e.g., 50+/−10 bases), about 60 bases (e.g., 60+/−10 bases), about 70 bases (e.g., 70+/−10 bases), about 90 bases (e.g., 90+/−10 bases), about 110 bases (e.g., 110+/−10 bases), about 130 bases (e.g., 130+/−10 bases), about 150 bases (e.g., 150+/−10 bases), about 170 bases (e.g., 170+/−10 bases), about 190 bases (e.g., 190+/−10 bases), about 210 bases (e.g., 210+/−10 bases), etc.

In some embodiments, the HD-FISH probe set comprises one or more subsets of 50 or more probes that bind to single 100 kb regions of sequenced human genomic DNA (i.e., the probe set contains 50 or more probes that bind within the same 100 kb region in the genome of the species). The probes may be 30-300 bases in length, including 40-300 bases in length and 70-300 bases in length. It is to be understood that each probe binds only to the targeted 100 kb region.

In some embodiments, the HD-FISH probe set comprises multiple subsets of 50 or more probes that, in aggregate, bind to 93% of 100 kb regions of the sequenced human genomic DNA (i.e., the probe set contains a number of probe subsets, each of which comprises at least 50 probes, which when used together bind to 93% of 100 kb regions of human genomic DNA that has been sequenced).

In some embodiments, the method further comprises synthesizing one or more subsets of probes within the HD-FISH probe set using an amplification reaction such as but not limited to a polymerase chain reaction (PCR).

In some embodiments, the method further comprises identifying the amplification primer pairs for each of the nucleic acid probes in the HD-FISH probe set.

In another aspect, the invention provides a composition comprising a probe set produced using any of the foregoing methods.

In another aspect, the invention provides a composition comprising one or more PCR-generated FISH probe sets each comprising 50 or more probes that bind within a contiguous 100 kb region of sequenced human genomic DNA.

In another aspect, the invention provides a composition comprising multiple PCR-generated FISH probe subsets, each comprising 50 or more probes, that in aggregate hybridize to 93% of discrete 100 kb regions of sequenced human genomic DNA.

In some embodiments, the probes are about 30-300 bases in length, 40-300 bases in length, or 70-300 bases in length. In some embodiments, the probes are relatively uniform in length (e.g., an average length +/−10 bases).

In some embodiments, the probes are fluorescently labeled. The probes may be uniformly labeled based on position of label and/or number of labels within the probe.

In some embodiments, the one or more probe subsets are produced using amplification reaction such as but not limited to a polymerase chain reaction on genomic DNA in the presence of an amplification primer probe set such as an HD-FISH probe primer set. In some embodiments, the one or more probe subsets are directly synthesized (e.g., using automated oligonucleotide synthesis independent of an enzyme-mediated reaction). In some embodiments, the probes may be uniformly fluorescently labeled during or after synthesis. In some embodiments, the probes may be fluorescently labeled at their 5′ ends (i.e., end-labeled at the 5′ end).

In some embodiments, the probes are single-stranded. In some embodiments, the probes are double-stranded.

In another aspect, the invention provides a composition comprising an HD-FISH probe primer set produced using any of the foregoing methods.

In another aspect, the invention provides a composition comprising an HD-FISH probe primer set that generates, through an amplification reaction, an HD-FISH probe set comprising one or more primer subsets, each having 50 or more probes, that together hybridize to 93% of discrete 100 kb regions of sequenced human genomic DNA.

In some aspects, primers within the primer set are fluorescently labeled (e.g., 5′ end-labeled).

In still another aspect, the invention provides a method comprising performing a fluorescent in situ hybridization (FISH) reaction in the presence of an HD-FISH probe set, or a subset thereof, that hybridizes to a 100 kb region of interest, wherein the probes are about 30 to about 300 bases in length (e.g., 40-300 bases in length or 70-300 bases in length) and wherein a FISH result that differs from a control indicates presence or absence of a chromosomal target such as a chromosomal abnormality.

In still another aspect, the invention provides a method comprising performing a fluorescent in situ hybridization (FISH) reaction in the presence of an HD-FISH probe set, or a subset thereof, that hybridizes to a 10 kb region of interest, wherein the probes are about 70 to about 300 bases in length, and wherein a FISH result that differs from a control indicates presence or absence of a chromosomal target such as a chromosomal abnormality.

In another aspect, the invention provides a method comprising performing a fluorescent in situ hybridization (FISH) reaction in the presence of an HD-FISH probe set, or a subset thereof, that hybridizes to a 3 kb region of interest, wherein the probes are about 70 to about 300 bases in length, and wherein a FISH result that differs from a control indicates presence or absence of a chromosomal target such as a chromosomal abnormality.

In some embodiments, the HD-FISH probe set or subset thereof comprises 5-30 probes. In some embodiments, the HD-FISH probe set or subset thereof comprises 10 probes. In some embodiments, the method further comprises synthesizing the HD-FISH probe set or a subset thereof using an amplification method such as PCR.

In some embodiments of the foregoing methods, the method is performed in more than 50% formamide (v/v), more than 60% formamide (v/v), or more than 70% formamide (v/v). In some embodiments, the method is performed in about 60-75% formamide, or about 60-70% formamide, or about 65-70% formamide. In some embodiments, the method is performed in the presence of SSC, dextran sulphate, phosphate buffer, EDTA and Denhardt's solution. In some embodiments, the method is performed in one or more of the following: 68% (v/v) formamide, 1.7×SSC, 10% dextran sulphate, 50 mM Na2HPO4/NaH2PO4, 1 mM EDTA, and 5×Denhardt's solution.

These and other aspects and embodiments of the invention will be described in greater detail herein.

### BRIEF DESCRIPTION OF DRAWINGS

FIG. 1 is a schematic illustrating the probe synthesis and labeling methods of the invention. (A) illustrates a ULS approach to labeling. (B) illustrates a 5′ end labeling via PCR followed by single-stranded probe preparation using biotinylated reverse primers incorporated during the PCR step.

FIG. 2 provides comparisons between labeling with commercially available HER2 probes and the probes of the invention. (A) shows images of interphase nucleic hybridized to a commercial HER2 probe that spans 450 kb of genomic DNA (left panel), and the HD-FISH dsDNA HER2 probes of the invention that span 40 kb (middle panel) and 3 kb (right panel) of genomic DNA. The commercial probe provides two strong signals which would be interpreted as the presence of two copies of the HER2 locus, while the HD-FISH probes of the invention provide four distinct signals (middle and right panels) suggesting that the HER2 locus has been duplicated. (B) shows the frequency distribution of dot widths when a probe set is used that consists of only 10 200-bp probes that bind within a 3 kb region of the genome. As shown, the majority of the dot widths observed are greater than the diffraction limit of the detection system.

FIG. 3 provides images of interphase nuclei hybridized to identically fluorescently labeled HD-FISH probes of the invention that target 10 loci on chromosome 17. The loci are separated from each other by at least about 8 megabases (Mb). The images show the presence of two or more clusters of signals within each interphase nucleus, with each cluster comprised of multiple distinct signals.

### DETAILED DESCRIPTION OF INVENTION

The invention provides, inter alia, methods for identifying and generating a FISH probe set and methods of using a FISH probe set of varying lengths. The probe sets of the invention are generated, in some instances, using an amplification method such as PCR. The invention therefore also provides a method for identifying optimal amplification primer pairs to be used to generate a probe set of interest. The invention also provides probe sets for use in the detection of virtually any nucleic acid region of interest, as well as amplification primer pairs for use in the generation of the probes of interest.

As described in greater detail herein, the probe set is generated by analyzing genomic DNA of a species. The analysis requires the sequence of the genomic DNA. As a result of various next-generation sequencing technologies, the genomic DNA sequences of many species are now available. The analysis may be performed to identify HD-FISH probes for an entire genome, or it may be performed to identify HD-FISH probes for a region of interest that is less than the entire genome. Such regions may be entire chromosomes or regions of chromosomes including an arm of a chromosome, or even specific genes within a chromosome (or genome). The region being targeted by the probes may be referred to herein as the region of interest.

The methods provided herein identify probes of any length (or size, as the terms are used interchangeably herein). Typically, shorter length probes are preferable since they are able to yield higher resolution and thus more detailed analysis of genomic alterations such as deletions, amplifications, translocations, and the like. As an example, a probe that is 100 kb or more in length may not be useful in detecting some genomic alterations because it binds to such a long region (i.e., mutations within that region may be overlooked provided the probe has sufficient complementarity to its binding site in its entirety).

In some embodiments, the probes range in length from about 30 bases to about 300 bases, including 40-300 bases and 70-300 bases. It is to be understood that the methods of the invention can be used to generate probes of any desired length. The probe length may be, without limitation, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, or about 300 bases.

It is to be understood that the probes of the invention may be provided as double-stranded nucleic acids (particularly when they are synthesized by an amplification reaction such as PCR) or as single-stranded nucleic acids (particularly when they are synthesized directly using for example automated synthesis techniques or as they may be rendered just prior to or during a FISH assay). As a result, the probe length may be referred to in terms of base pairs (bp) or simply as bases.

The methods can be used to generate probe sets comprising probes of relatively uniform length. Such probes may therefore differ in length from each other by about 40, or about 30 or about 20, or about 10, or about 5 bases. As an example, a probe set may comprise probes that are 210+/−10 bases in length (or in other words, may have a length range of 200-220 bases).

The methods can also be used to generate probe sets comprising probes that are relatively uniform in their binding affinity to their targets. This is important in a FISH assay because all the probes will be expected to bind to their targets under the same hybridization conditions with the same affinity. If one or more probes are better able to bind to their targets than others, then the results and existing mutations may be obscured resulting in false negatives or false positives.

Thus, the methods of the invention can be used to generate an optimal probe set having relatively uniform length and relatively uniform binding affinity under certain hybridization conditions. The invention provides particular hybridization conditions that were used to perform FISH assays using probes of various lengths, including 200-mers, 100-mers, and 40-mers.

The Examples demonstrate the use of the methods and compositions of the invention in a robust HD-FISH assay. These findings were further confirmed as evidenced by Bienko et al. Nature Methods 2013 February; 10(2):122-4, the entire contents of which are incorporated by reference herein.

The methods to produce an HD-FISH probe set are typically multi-step processes. Below is a description of one such process.

In step (a), a plurality of nucleic acids is identified, each having a nucleotide sequence that is unique relative to the sequenced genomic DNA of a species. This step is aimed at identifying the maximum number of sequences having a certain degree of uniqueness in a region of interest. The region of interest may be the entire genome or a region within that genome including a specific gene. “Sequences” in this context is used synonymously with the nucleic acids being identified. “Unique” in the context of at least this step means at a minimum that the sequence has no other exact match (i.e., 100% identity) in the entire sequenced genome of the species. In addition, it also means that the sequence does not share more than a certain degree of identity with any other sequence in the entire sequenced genome of the species. The cut-off for this initial degree of identity may vary depending on the genome and its complexity. In a human genome, the cut-off may be 80% or 90%. A cut-off of 80% means that a sequence having equal to or more than 80% identity with another sequence over 100 or more base pairs outside of its own region in the genome is excluded from the analysis at this step. Similarly, at a cut-off of 80%, a sequence having less than 80% identity with another sequence of relatively equal length in the genome is maintained and further interrogated using steps (b) through (d). The “sequenced genomic DNA of a species” refers to the known genomic sequence of a species. Known genomic DNA sequences are publicly available in various databases including NCBI databases, Human Genome Project databases, the UCSC genome databases, Ensembl databases, and the like, most or all of which are available online.

Step (a) may be performed by first generating a number of sequences for the region of interest starting at one position (typically an end of the region) and moving along the region at a particular interval. As an example, sequences that are 500 bases in length may be generated by starting at one end and moving along the region of interest every 100 bases. Each sequence will therefore overlap with certain other sequences. The length of the sequence and the distance between starting points of consecutive sequences may vary from these lengths. For example, the sequences may be 200, 300, 400, 500, 600 or 700 bases in length and the starting points may be spaced apart by 40, 60, 80, 100, 120 or 140 bases provided that neighboring sequences overlap with each other.

Step (a) may reveal unique overlapping sequences. In this case, the method may also comprise a step of generating a new sequence consisting of the unique overlapping sequences. The combined sequence, rather than the individual overlapping sequences, may then be used in step (b).

In step (b), one or more nucleic acid fragments ranging in length from about 30 to about 300 bases is identified from each unique nucleic acid (or sequence) identified in step (a). This step may be performed by limiting the length of probe that is desired. For example, it may be performed to identify fragments that are about 40 bases (e.g., 40+/−5 bases) or 70 bases (e.g., 70+/−10 bases) in length. These nucleic acid fragments are considered “putative probes” since it is from this subset of nucleic acids that the final probe set will be selected.

Once the fragments (or putative probes) are identified, so too are the amplification primer pairs that can be used to generate (e.g., amplify) each fragment. There may be a single primer pair for a nucleic acid fragment or there may be more than one primer pair for a nucleic acid fragment, depending on the length of the fragment. The primer length may vary. In some embodiments, the primer length ranges from about 18-22 base pairs. The total number of primer pairs per fragment may vary depending on the length of the fragment and the length of the desired probe. For example, a 500 base pair fragment can give rise to two probes that are 200 base pairs each in length or three or four probes that are 100 base pairs each in length. Primer sequence complexity and length will be generally uniform across a primer set. This is particularly true for primers that will amplified under the same amplification conditions including in the same reaction so that the likelihood of amplification is generally the same for all primer pairs in the set.

It is to be understood that step (b) may be carried out in reverse order, such that the primer pairs are first identified followed by identification of the putative probes. In this instance, the primer pairs are still selected based on the desired length of the probe. For example, if probes having a length of about 200 bases are desired, then primer pairs that yield about 200 bp fragments are chosen. An initial set of primer pairs may be generated by starting at an end of a nucleic acid sequence and moving along the sequence by certain intervals, in a manner similar to that of step (a). In some embodiments, there is no sequence overlap between primers. In some embodiments, there is no sequence overlap between the nucleic acid fragments that would be generated using the primer pairs. In some embodiments, there is at least one base pair between adjacent probes. Step (b) therefore identifies a putative probe set and a putative primer set.

The subsequent steps are directed towards excluding fragments from the putative probe set, thereby ultimately arriving at a final probe set that may be used in HD-FISH.

In step (c), a nucleic acid fragment (or putative probe) is eliminated from the putative probe set of (b) if the amplification primer pair used to amplify the nucleic acid fragment also amplifies other fragments. Step (c) therefore analyzes the ability of a primer pair to amplify one or more fragments from a nucleic acid sample. The primer pair must amplify only its associated fragment; if it fails to do so the pair and its associated fragment are excluded from the putative primer set and the putative probe set respectively. As an example, if the primer pair amplifies more than one fragment, then the pair and its associated fragment are excluded. If the primer pair amplifies a single fragment that is not its associated fragment, then the pair and its associated fragment are excluded.

Step (c) may be performed in the context of the entire genome or in the context of a limited region of the genome. For example, if the primer pair will ultimately be used to generate probes from a sample that comprises genomic DNA, then the analysis in step (c) should be carried out in the context of the complete genome of the species. However, if the primer pair will ultimately be used to generate probes from a limited region of the genome, then the analysis in step (c) should be carried out in the context of that limited region of the genome. This latter approach may be used if the probes were generated from, for example, a bacterial artificial chromosome (BAC). To be clear, the probes may be generated from a limited nucleic acid sample (e.g., a BAC) yet they may be used to screen a genomic sample such as an entire interphase nucleus or a complete set of metaphase chromosomes.

In step (d), nucleic acid fragments from (b) are screened for their uniqueness relative to other regions of genomic DNA of the species. One way of testing this is to determine if the fragment binds to more than one region in the genome. This screening is more stringent than that carried out in step (a). As an example, if the identity cut-off is set at 80% in step (a), then the identity cut-off is set to, for example, 70% in step (d). As described above, if the cut-off is 70%, then a nucleic acid fragment having equal to or more than 70% identity to a sequence in genomic DNA (other than itself) is excluded from the putative probe set. Under the same criteria, a nucleic acid fragment having less than 70% identity to sequences in genomic DNA are maintained in the putative probe set.

Steps (c) and (d) can be performed in either order (i.e., step (c) may be performed before or after step (d)).

It is to be understood that once the probes of interest are identified, so too are the amplification primer pairs that will amplify the probes of interest. This is because the method identifies suitable probes by analyzing not only the probe sequence and how likely it is to bind to other spurious regions in the genome, but also by analyzing the amplification primer pair and how likely it is to amplify other spurious regions from a genomic sample. Thus, the same method can be used to identify probes and/or their respective amplification primer pairs. It is also to be understood that the method may be used to identify a probe set for an entire genome or a probe set for a particular region of a genome, and similarly a primer set for an entire genome or a primer set for a particular region of the genome.

It is to be understood that the probes so identified may thereafter be generated in an amplification-dependent (e.g. PCR reaction) or an amplification-independent (e.g., direct synthesis) manner.

Another method provided by the invention involves step (a) and step (d) as recited above. Thus, the invention contemplates the identification of probes in the absence of identification of primers to amplify such probes.

The methods can be performed entirely with a computer and/or via a computer simulation and/or algorithm (e.g., in silico). Such computer simulations allow an end user to set various parameters such as length of the nucleic acids originally identified (e.g., in step (a)), length of probes and putative probes and thus placement of amplification primer pairs, degree of stringency to be applied when identifying unique sequences, and the like. The degree of stringency may be set as a percentage of identity. As an example, a combination of stringency (e.g., 80% cut-off) and a probe length of 200 or 300 base pairs may be used.

Steps (a) and (d) may be performed by comparing the generated nucleic acid sequences against a genomic sequence database. Computer programs such as BLAST and BLAT can be used for this purpose. BLAST is available from the NCBI website and BLAT is available from the online Ohio Supercomputer Center. Step (b) may be performed using computer programs such as Primer3. The Primer3 program is available online from a number of academic institutions including but not limited to Massachusetts Institute of Technology. Customized scripts for primer design may also be used. Step (c) may be performed using computer programs such as e-PCR. The e-PCR program is available online from NCBI. Customized scripts may also be used. It is also to be understood that one or more than one steps of the method (including all the steps of the method) may be performed manually.

Thus in some instances the method may be performed by a computer specifically programmed to carry out the step (a) through (d) described above or step (a) and step (d) only as described above. For example, the methods are performed by a computer that includes a processing module that carries out the method. The method may therefore be a computer-implemented method. In certain embodiments, the method is coded onto a computer-readable medium in the form of “programming”, where the term “computer storage readable medium” as used herein refers to any storage medium that participates in providing instructions and/or data to a computer for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external to the computer.

Whether performed in silico or manually, the method can be followed by actual synthesis of the probe set. Synthesis of the probe may employ the primer set in an amplification reaction such as PCR. The amplification method can be used to label the probes with detectable labels such as fluorophores. This may be done by performing the amplification reaction in the presence of labeled nucleotides that are incorporated into the synthesized strands of DNA. As an example, all of a subset of nucleotides may be detectable labeled. When more than one type of nucleotide is labeled, all nucleotides are typically labeled with the same fluorophore (e.g., all labeled nucleotides are labeled with a FITC fluorophore). The probes may also be labeled uniformly by performing the amplification reaction using primers that are labeled. The labeled primer may be the forward or the reverse primer. It should be appreciated that the methods of the invention allow directed labeling of a specific strand of a probe by labeling either the forward or the reverse primer. This labeling flexibility is not typically available using FISH probes of the prior art. Primers may be labeled at their ends (e.g., 5′ end), or they may be labeled internally, or they may be labeled throughout. Preliminary experiments indicate that 5′ end labeling is sufficient. The primers may be labeled directly (i.e., they may be directly conjugated to a fluorophore) or they may be labeled indirectly (e.g., they may be biotinylated and then reacted with an avidin or streptavidin conjugated fluorophore.

Other means of attaching labels to nucleic acids are known in the art and include, for example, nick translation or end-labeling, and phosphorylation of the nucleic acid and subsequent attachment of a nucleic acid linker joining the oligonucleotides to a label. In certain embodiments, the FISH probes may be labeled by Universal Linkage System (ULS™ KREATECH Diagnostics). In brief, ULS™ labeling is based on the stable binding properties of platinum (II) to nucleic acids. The ULS™ molecule consists of a mono-functional platinum complex coupled to a detectable molecule of choice. Standard methods may be used for labeling the oligonucleotide, for example, as set out in Ausubel, et al. (Short Protocols in Molecular Biology, 3rd ed., Wiley & Sons, 1995) and Sambrook, et al. (Molecular Cloning: A Laboratory Manual, Third Edition, (2001) Cold Spring Harbor, N.Y.).

It is also to be understood that the probes of the invention may be synthesized directly rather than through an enzyme-mediated process such as PCR. Such directly synthesized probes may be labeled during synthesis or post-synthesis. Methods for such labeling are known in the art and reference may be made for example to U.S. Pat. Nos. 4,997,928, 5,262,536, 5,688,940 and 7,705,150, which evidence the ability to label nucleic acids.

The following is an example of how one method of the invention may be carried out. It is to be understood that this example is merely illustrative and that one of ordinary skill in the art with knowledge of the teachings provided herein will be able to vary the method within the spirit of the invention.

This example identifies probes for the entire human genome. Briefly, the sequence of each human chromosome is split into 500 bp tiled fragments, sliding at 100 bp intervals. Each fragment is then searched against a known human genome sequence using a computer program such as BLAT. Unique fragments are identified and, if overlapping, they are merged together. A computer program such as Primer3 is used to generate tiled primer pairs based on the unique fragments. Each primer pair so generate is then checked for uniqueness using a computer program such as e-PCR. Each unique fragment is again checked for uniqueness against the human genome using a more sensitive search such as a more sensitive BLAT search.

More specifically, each of the 23 human chromosomes is broken into, in this example, 500 bp fragments, sliding along the chromosome in 100 bp steps. This results in 250 million overlapping 500 bp fragments. Each 500 bp fragment is searched against the human genome with the BLAT software. The BLAT homology search finds matches in the genome with 80% or higher similarity, in this example. Fragments with non-self matches are discarded from further consideration. Unique 500 bp fragments are merged (or “stitched”) together to create contiguous blocks of non-repetitive DNA. Primer3 software is used to design primer pairs, separated, in this example, by 200-220 bp, tiled along the unique DNA stretches. Each primer pair identifies a potential probe for DNA-FISH. Default Primer3 parameters are used to pick the primer pairs. The Examples provided herein used the default Primer3 parameters with a target probe length set to 70, 100 or 200 base pairs. This resulted in 5,120,725 total primer pairs. To avoid cross-amplification, each primer pair is screened for specificity with e-PCR software. Mismatches of 0-2 bp per primer are permitted, in some instances. As an example, if 20/22 bases of the forward primer and the reverse primer are identical to another region, then the primer pair is excluded. Primer pairs that have non-self products are discarded. As an example, primer pairs that amplify another region, up to for example 500 base pairs, are excluded. This resulted in the exclusion of 40,705 primer pairs (and putative probes). The DNA sequences flanked by the primer pairs (i.e., the probes) are individually screened for uniqueness by another round of BLAT searching against the human genome. Probes with matches of 70% or higher identity outside of their own region are discarded. This further excluded 256,236 putative probes. The final probe count in the resultant probe set is 4,823,784. This probe set covers over 93% of the 2.9 Gb of sequenced human genomic DNA. Thus on average the probe set provides more than 50 probes per 100 kb of sequenced human genomic DNA.

Using the methods of the invention, probes specific for single genes have been generated. Examples of single gene targets include those provided below in Table 1. Single gene HD-FISH probes can therefore be prepared that are specific for genes such as ERBB2/HER2, MET, EGFR, BCR, ABL1, PML, RARalpha, PTEN, and RPS14. Alterations in each of these genes has been used to diagnose and/or prognose a particular condition. The alterations include but are not limited to amplifications (e.g., HER2 in breast cancer) and rearrangements and translocations (e.g., BCR/ABL1 and PML/RARα, both in leukemia). Other alterations are described in the Tables. The art is familiar with the use of these genes in diagnostic and prognostic testing.

TABLE 1

| GENE(S) | ABERRATION | DISEASE | IMPORTANCE |
|---|---|---|---|
| ERBB2/HER2 | amp(17)(q2.1) | Breast cancer | Patient Selection for |
| | | | Trastuzumab |
| MET | amp(7)(q31) | Breast cancer | Identification of patients |
| | | | possibly resistant to |
| | | | Trastuzumab |
| EGFR | amp(7)(p12) | Breast, colon, lung, | Patient selection for |
| | | head-and-neck cancer | Cetuximab and |
| | | | Panitumumab. |
| | | | Identification of patients |
| | | | possibly resistant to |
| | | | Trastuzumab |
| BCR; ABL1 | t(9; 22)(q34.1; q11.23) | Chronic myeloid | Patient selection for |
| | | leukemia | Imatinib, Dasatinib, and |
| | | | Nilotinib |
| PML; RARalpha | t(15; 17)(q22; q21) | Acute promyelocytic | Patient selection for |
| | | leukemia | ATRA and arsenic |
| | | | trioxide |
| PTEN | del(10)(q23.3) | Breast cancer | Identification of patients |
| | | | possibly resistant to |
| | | | Trastuzumab |
| RPS14 | del(5)q32) | Myelodysplastic | Patient selection for |
| | | syndrome (5q minus | Lenalidomide |
| | | syndrome) | |
| EML4; ALK | t(2; 2)(2p21; 2p23); | Lung cancer | Patient selection for |
| | inv(2) | | Crizotinib |

The methods can also be used to generate “spotting” HD-FISH probes that detect a chromosome or a chromosomal region (such as a chromosomal arm) as a cloud of dots. Examples of such chromosomes or chromosomal regions include those provided below in Table 2. These spotting probe sets may consist of a variable number of probes in defined genes known to have low copy number variation across different diseases. Accordingly, alteration in the level of staining in these chromosomes can be used for diagnostic or prognostic assessments.

TABLE 2

| CHROMOSOME | ABERRATION | DISEASE | IMPORTANCE |
|---|---|---|---|
| 1p | del(1p) | Anaplastic | Diagnosis of gliomas |
| | | Oligodendroglioma | |
| 5q | del(5q) | Myelodysplastic syndromes | Diagnosis of myeloid |
| | | and acute myeloid leukemia | leukemias |
| 7q | del(7q) | Acute myeloid leukemia | Diagnosis of myeloid |
| | | | leukemias |
| 8 | +8 | Acute myeloid leukemia | Diagnosis of myeloid |
| | | | leukemias |
| 12 | +12 | Chronic lymphocytic | Diagnosis of lymphatic |
| | | leukemia | leukemias |
| 13 | +13 | Patau syndrome | Pre-natal screening |
| 18 | +18 | Edwards syndrome | Pre-natal screening |
| 19q | del(19q) | Anaplastic | Diagnosis of gliomas |
| | | oligodendroglioma | |
| 20q | del(20q) | Myelodysplastic syndromes, | Diagnosis of leukemias |
| | | acute myeloid leukemia, | |
| | | and chromic | |
| | | myeloproliferative diseases | |
| 21 | +21 | Down syndrome | Pre-natal screening |

Provided below in Table 2A are the SEQ ID NOs for exemplary primer sets for generating probes that bind to the genes and chromosomal regions listed in Tables 1 and 2. The corresponding sequence listing is provided and incorporated herewith, and is to be considered part of the application. It will be understood in view of the invention that the primers themselves may be used as probes or they may be used to generate probes of longer length. All or a subset of primer pairs within a primer set for a particular gene or chromosomal region of interest may be used. Ensembl gene IDs are provided for each primer set for a gene. For spotting probes, each primer set contains the list of primers for a chromosome or arm (e.g., 5q, 7q, 19q for long arms of chromosomes 5, 7, 19). Primer pairs in each set are numbered progressively from the telomere of the short arm towards the telomere of the long arm.

TABLE 2A

| Gene Name | Forward Primers* | Reverse Primers* |
|---|---|---|
| ABL1 | SEQ ID NOs: 1-264 | SEQ ID NOs: 265-528 |
| ID: ENSG00000097007 | | |
| BCR | SEQ ID NOs: 529-784 | SEQ ID NOs: 785-1040 |
| ID: ENSG00000186716 | | |
| EGFR | SEQ ID NOs: 1041-1652 | SEQ ID NOs: 1653-2264 |
| ID: ENSG00000146648 | | |
| HER2 3 kb probe | SEQ ID NOs: 2265-2279 | SEQ ID NOs: 2280-2294 |
| HER2 40 kb probe | SEQ ID NOs: 2295-2349 | SEQ ID NOs: 2350-2404 |
| MET | SEQ ID NOs: 2405-2701 | SEQ ID NOs: 2702-2998 |
| ID: ENSG00000105976 | | |
| PML | SEQ ID NOs: 2999-3108 | SEQ ID NOs: 3109-3218 |
| ID: ENSG00000140464 | | |
| PTEN | SEQ ID NOs: 3219-3422 | SEQ ID NOs: 3423-3626 |
| ID: ENSG00000171862 | | |
| RARα | SEQ ID NOs: 3627-3756 | SEQ ID NOs: 3757-3886 |
| ID: ENSG00000131759 | | |
| RPS14 | SEQ ID NOs: 3887-3967 | SEQ ID NOs: 3968-4048 |
| ID: ENSG00000164587 | | |

Chr5q spotting probe

| | | |
|---|---|---|
| Chr5q-1 | SEQ ID NOs: 4049-4144 | SEQ ID NOs: 4145-4240 |
| Chr5q-2 | SEQ ID NOs: 4241-4336 | SEQ ID NOs: 4337-4432 |
| Chr5q-3 | SEQ ID NOs: 4433-4528 | SEQ ID NOs: 4529-4624 |
| Chr5q-4 | SEQ ID NOs: 4625-4720 | SEQ ID NOs: 4721-4816 |
| Chr5q-5 | SEQ ID NOs: 4817-4912 | SEQ ID NOs: 4913-5008 |
| Chr5q-6 | SEQ ID NOs: 5009-5104 | SEQ ID NOs: 5105-5200 |
| Chr5q-7 | SEQ ID NOs: 5201-5296 | SEQ ID NOs: 5297-5392 |
| Chr5q-8 | SEQ ID NOs: 5393-5488 | SEQ ID NOs: 5489-5584 |
| Chr5q-9 | SEQ ID NOs: 5585-5680 | SEQ ID NOs: 5681-5776 |
| Chr5q-10 | SEQ ID NOs: 5777-5872 | SEQ ID NOs: 5873-5968 |
| Chr5q-11 | SEQ ID NOs: 5969-6064 | SEQ ID NOs: 6065-6160 |
| Chr5q-12 | SEQ ID NOs: 6161-6256 | SEQ ID NOs: 6257-6352 |
| Chr5q-13 | SEQ ID NOs: 6353-6448 | SEQ ID NOs: 6449-6544 |

Chr7q spotting probe

| | | |
|---|---|---|
| Chr7q-1 | SEQ ID NOs: 6545-6640 | SEQ ID NOs: 6641-6736 |
| Chr7q-2 | SEQ ID NOs: 6737-6832 | SEQ ID NOs: 6833-6928 |
| Chr7q-3 | SEQ ID NOs: 6929-7024 | SEQ ID NOs: 7025-7120 |
| Chr7q-4 | SEQ ID NOs: 7121-7216 | SEQ ID NOs: 7217-7312 |
| Chr7q-5 | SEQ ID NOs: 7313-7408 | SEQ ID NOs: 7409-7504 |
| Chr7q-6 | SEQ ID NOs: 7505-7600 | SEQ ID NOs: 7601-7696 |
| Chr7q-7 | SEQ ID NOs: 7697-7792 | SEQ ID NOs: 7793-7888 |
| Chr7q-8 | SEQ ID NOs: 7889-7984 | SEQ ID NOs: 7985-8080 |
| Chr7q-9 | SEQ ID NOs: 8081-8176 | SEQ ID NOs: 8177-8272 |

Chr8 spotting probe

| | | |
|---|---|---|
| Chr8-1 | SEQ ID NOs: 8273-8368 | SEQ ID NOs: 8369-8464 |
| Chr8-2 | SEQ ID NOs: 8465-8560 | SEQ ID NOs: 8561-8656 |
| Chr8-3 | SEQ ID NOs: 8657-8752 | SEQ ID NOs: 8753-8848 |
| Chr8-4 | SEQ ID NOs: 8849-8944 | SEQ ID NOs: 8945-9040 |
| Chr8-5 | SEQ ID NOs: 9041-9136 | SEQ ID NOs: 9137-9232 |
| Chr8-6 | SEQ ID NOs: 9233-9328 | SEQ ID NOs: 9329-9424 |
| Chr8-7 | SEQ ID NOs: 9425-9520 | SEQ ID NOs: 9521-9616 |
| Chr8-8 | SEQ ID NOs: 9617-9712 | SEQ ID NOs: 9713-9808 |
| Chr8-9 | SEQ ID NOs: 9809-9904 | SEQ ID NOs: 9905-10000 |
| Chr8-10 | SEQ ID NOs: 10,001-10096 | SEQ ID NOs: 10,097-10192 |
| Chr8-11 | SEQ ID NOs: 10193-10288 | SEQ ID NOs: 10289-10384 |
| Chr8-12 | SEQ ID NOs: 10385-10480 | SEQ ID NOs: 10481-10576 |
| Chr8-13 | SEQ ID NOs: 10577-10672 | SEQ ID NOs: 10673-10768 |
| Chr8-14 | SEQ ID NOs: 10769-10864 | SEQ ID NOs: 10865-10960 |
| Chr8-15 | SEQ ID NOs: 10961-11056 | SEQ ID NOs: 11057-11152 |

Chr12 spotting probe

| | | |
|---|---|---|
| Chr12-1 | SEQ ID NOs: 11153-11248 | SEQ ID NOs: 11249-11344 |
| Chr12-2 | SEQ ID NOs: 11345-11440 | SEQ ID NOs: 11441-11536 |
| Chr12-3 | SEQ ID NOs: 11537-11632 | SEQ ID NOs: 11633-11728 |
| Chr12-4 | SEQ ID NOs: 11729-11824 | SEQ ID NOs: 11825-11920 |
| Chr12-5 | SEQ ID NOs: 11921-12016 | SEQ ID NOs: 12017-12112 |
| Chr12-6 | SEQ ID NOs: 12113-12208 | SEQ ID NOs: 12209-12304 |
| Chr12-7 | SEQ ID NOs: 12305-12400 | SEQ ID NOs: 12401-12496 |
| Chr12-8 | SEQ ID NOs: 12497-12592 | SEQ ID NOs: 12593-12688 |
| Chr12-9 | SEQ ID NOs: 12689-12784 | SEQ ID NOs: 12785-12880 |
| Chr12-10 | SEQ ID NOs: 12881-12976 | SEQ ID NOs: 12977-13072 |
| Chr12-11 | SEQ ID NOs: 13073-13168 | SEQ ID NOs: 13169-13264 |
| Chr12-12 | SEQ ID NOs: 13265-13360 | SEQ ID NOs: 13361-13456 |
| Chr12-13 | SEQ ID NOs: 13457-13552 | SEQ ID NOs: 13553-13648 |

Chr13 spotting probe

| | | |
|---|---|---|
| Chr13-1 | SEQ ID NOs: 13649-13744 | SEQ ID NOs: 13745-13840 |
| Chr13-2 | SEQ ID NOs: 13841-13936 | SEQ ID NOs: 13937-14032 |
| Chr13-3 | SEQ ID NOs: 14033-14128 | SEQ ID NOs: 14129-14224 |
| Chr13-4 | SEQ ID NOs: 14225-14320 | SEQ ID NOs: 14321-14416 |
| Chr13-5 | SEQ ID NOs: 14417-14512 | SEQ ID NOs: 14513-14608 |
| Chr13-6 | SEQ ID NOs: 14609-14704 | SEQ ID NOs: 14705-14800 |
| Chr13-7 | SEQ ID NOs: 14801-14896 | SEQ ID NOs: 14897-14992 |
| Chr13-8 | SEQ ID NOs: 14993-15088 | SEQ ID NOs: 15089-15184 |
| Chr13-9 | SEQ ID NOs: 15185-15280 | SEQ ID NOs: 15281-15376 |
| Chr13-10 | SEQ ID NOs: 15377-15472 | SEQ ID NOs: 15473-15568 |

Chr17 spotting probe

| | | |
|---|---|---|
| Chr17-1 | SEQ ID NOs: 15569-15623 | SEQ ID NOs: 15624-15678 |
| Chr17-2 | SEQ ID NOs: 15679-15733 | SEQ ID NOs: 15734-15788 |
| Chr17-3 | SEQ ID NOs: 15789-15844 | SEQ ID NOs: 15845-15900 |
| Chr17-4 | SEQ ID NOs: 15901-15956 | SEQ ID NOs: 15957-16012 |
| Chr17-5 | SEQ ID NOs: 16013-16067 | SEQ ID NOs: 16068-16122 |
| Chr17-6 | SEQ ID NOs: 16123-16175 | SEQ ID NOs: 16176-16228 |
| Chr17-7 | SEQ ID NOs: 16229-16283 | SEQ ID NOs: 16284-16338 |
| Chr17-8 | SEQ ID NOs: 16339-16392 | SEQ ID NOs: 16393-16446 |
| Chr17-9 | SEQ ID NOs: 16447-16500 | SEQ ID NOs: 16501-16554 |
| Chr17-10 | SEQ ID NOs: 16555-16607 | SEQ ID NOs: 16608-16660 |

Chr18 spotting probe

| | | |
|---|---|---|
| Chr18-1 | SEQ ID NOs: 16661-16756 | SEQ ID NOs: 16757-16852 |
| Chr18-2 | SEQ ID NOs: 16853-16948 | SEQ ID NOs: 16949-17044 |
| Chr18-3 | SEQ ID NOs: 17045-17140 | SEQ ID NOs: 17141-17236 |
| Chr18-4 | SEQ ID NOs: 17237-17332 | SEQ ID NOs: 17333-17428 |
| Chr18-5 | SEQ ID NOs: 17429-17524 | SEQ ID NOs: 17525-17620 |
| Chr18-6 | SEQ ID NOs: 17621-17716 | SEQ ID NOs: 17717-17812 |
| Chr18-7 | SEQ ID NOs: 17813-17908 | SEQ ID NOs: 17909-18004 |
| Chr18-8 | SEQ ID NOs: 18005-18100 | SEQ ID NOs: 18101-18196 |

Chr19q spotting probe

| | | |
|---|---|---|
| Chr19q-1 | SEQ ID NOs: 18197-18292 | SEQ ID NOs: 18293-18388 |
| Chr19q-2 | SEQ ID NOs: 18389-18484 | SEQ ID NOs: 18485-18580 |
| Chr19q-3 | SEQ ID NOs: 18581-18676 | SEQ ID NOs: 18677-18772 |
| Chr19q-4 | SEQ ID NOs: 18773-18868 | SEQ ID NOs: 18869-18964 |
| Chr19q-5 | SEQ ID NOs: 18965-19060 | SEQ ID NOs: 19061-19156 |
| Chr19q-6 | SEQ ID NOs: 19157-19252 | SEQ ID NOs: 19253-19348 |

Chr20q spotting probe

| | | |
|---|---|---|
| Chr20q-1 | SEQ ID NOs: 19349-19444 | SEQ ID NOs: 19445-19540 |
| Chr20q-2 | SEQ ID NOs: 19541-19636 | SEQ ID NOs: 19637-19732 |
| Chr20q-3 | SEQ ID NOs: 19733-19828 | SEQ ID NOs: 19829-19924 |
| Chr20q-4 | SEQ ID NOs: 19925-20020 | SEQ ID NOs: 20021-20116 |
| Chr20q-5 | SEQ ID NOs: 20117-20212 | SEQ ID NOs: 20213-20308 |
| Chr20q-6 | SEQ ID NOs: 20309-20404 | SEQ ID NOs: 20405-20500 |
| Chr20q-7 | SEQ ID NOs: 20501-20596 | SEQ ID NOs: 20597-20692 |

Chr21 spotting probe

| | | |
|---|---|---|
| Chr21-1 | SEQ ID NOs: 20693-20788 | SEQ ID NOs: 20789-20884 |
| Chr21-2 | SEQ ID NOs: 20885-20980 | SEQ ID NOs: 20981-21076 |
| Chr21-3 | SEQ ID NOs: 21077-21172 | SEQ ID NOs: 21173-21268 |
| Chr21-4 | SEQ ID NOs: 21269-21364 | SEQ ID NOs: 21365-21460 |
| Chr21-5 | SEQ ID NOs: 21461-21556 | SEQ ID NOs: 21557-21652 |
| Chr21-6 | SEQ ID NOs: 21653-21748 | SEQ ID NOs: 21749-21844 |
| Chr21-7 | SEQ ID NOs: 21845-21940 | SEQ ID NOs: 21941-22036 |
| ALK1 | SEQ ID NOs: 22037-23759 | SEQ ID NOs: 23760-25482 |
| ID: ENSG00000171094 | | |
| EML4 | SEQ ID NOs: 25483-25781 | SEQ ID NOs: 25782-26080 |
| ID: ENSG00000143924 | | |

Chr1p spotting probe

| | | |
|---|---|---|
| Chr1p-1 | SEQ ID NOs: 26081-26176 | SEQ ID NOs: 26177-26272 |
| Chr1p-2 | SEQ ID NOs: 26273-26368 | SEQ ID NOs: 26369-26464 |
| Chr1p-3 | SEQ ID NOs: 26465-26560 | SEQ ID NOs: 26561-26656 |
| Chr1p-4 | SEQ ID NOs: 26657-26752 | SEQ ID NOs: 26753-26848 |
| Chr1p-5 | SEQ ID NOs: 26849-26944 | SEQ ID NOs: 26945-27040 |
| Chr1p-6 | SEQ ID NOs: 27041-27136 | SEQ ID NOs: 27137-27232 |
| Chr1p-7 | SEQ ID NOs: 27233-27328 | SEQ ID NOs: 27329-27424 |
| Chr1p-8 | SEQ ID NOs: 27425 27520 | SEQ ID NOs: 27521-27616 |
| Chr1p-9 | SEQ ID NOs: 27617-27712 | SEQ ID NOs: 27713-27808 |
| Chr1p-10 | SEQ ID NOs: 27809-27904 | SEQ ID NOs: 27905-28000 |
| Chr1p-11 | SEQ ID NOs: 28001-28096 | SEQ ID NOs: 28097-28192 |
| Chr1p-12 | SEQ ID NOs: 28193-28288 | SEQ ID NOs: 28289-28384 |

*It is to be understood that there is one reverse primer for each forward primer. The sequence listing provides all the forward primers for a given gene or chromosome and then all the reverse primers for a given gene or chromosome. For each gene or chromosome, the first forward primer (first forward primer SEQ ID NO:) pairs with the first reverse primer (first reverse primer SEQ ID NO:), the second forward primer (second forward primer SEQ ID NO:) pairs with the second reverse primer (second reverse primer SEQ ID NO:), and so on throughout the primer set, such that the last forward primer (last forward primer SEQ ID NO:) pairs with the last reverse primer (last reverse primer SEQ ID NO:).

Accordingly, the invention also provides probe sets and primer sets that are useful in HD-FISH, and compositions and kits comprising one or both of these sets.

The HD-FISH probe set may comprise probes that are about 30-300 bases in length. It is to be understood that probes of these various lengths may be identified by performing the methods of the invention (and thereby setting a desired length range for the identified probes). Alternatively, probes of a desired length may be generated based on the sequences of longer probes. For example, the methods of the invention may be performed to identity probes that are 200 bases in length. Such probes may be used to generate probes that are 100 bases in length (e.g., by selecting a contiguous 100 base sequence therein such as without limitation the 5′ most 100 bases, or the middle 100 bases, or the 3′ most 100 bases).

The HD-FISH probe set may comprise probes that are labeled with detectable labels. Detectable labels include but are not limited to fluorophores. The nature of the detectable label will be governed by the detection system used to detect the probe binding events. In most instances, fluorophores are the label of choice. The primers and probes may also be coupled to biotin, as an example, and the locus may be visualized after hybridization using Tyramide Signal Amplification (TSA™, from Perkin Elmer).

The probes may be uniformly labeled. Uniform labeling refers to similar (or identical) location and amount of label between probes. An example of uniform labeling is 5′ end-labeled. Uniform labeling of this nature may be achieved using PCR and uniformly labeled forward or reverse primers.

The probes may be single or double-stranded.

In some instances, the HD-FISH probe set comprises one or more subsets of probes that bind to a single (i.e., contiguous) 100 kb region of sequenced human genomic DNA. A probe subset may comprise 50 or more probes.

In some instances, the HD-FISH probe set comprises multiple probe subsets that, in aggregate, bind to 93% of 100 kb regions of sequenced human genomic DNA. A probe subset may comprise 50 or more probes.

The invention also provides HD-FISH primer sets that amplify an HD-FISH probe set. The comprising one or more primer pair subsets each having 50 or more probes that together hybridize to 93% of discrete 100 kb regions of sequenced human genomic DNA.

The invention also provides a method for performing FISH using the probe sets and/or the primer sets described herein. The method comprises performing FISH in the presence of an HD-FISH probe set, or a subset thereof, that hybridizes to a 10 kb region of interest, wherein the probes are about 30 to about 300 bases including about 40 to about 300 bases in length, and wherein a FISH result that differs from a control indicates a chromosomal abnormality.

The invention provides another method for performing FISH using the probe sets and/or the primer sets described herein. The method comprises performing FISH in the presence of an HD-FISH probe set, or a subset thereof, that hybridizes to a 3 kb region of interest, wherein the probes are about 70 to about 300 bases in length, and wherein a FISH result that differs from a control indicates a chromosomal abnormality.

In some instances, the HD-FISH probe set or subset thereof comprises 5-30 probes. The HD-FISH probe set or subset thereof may comprise 10 probes. In some embodiments, a set or subset may comprise 5, 10, 15, 20, 25, 30, 35, 40, or 50 probes.

In some embodiments, the invention contemplates synthesizing the HD-FISH probe set or a subset thereof using an amplification method such as a PCR reaction. Single-stranded probes, including single-stranded DNA probes, can also be obtained commercially from sources such as Bioneer (Extendamers™), IDT (Ultramer™), and Biosearch Technologies. Based on the teachings provided herein including but not limited to the primer pairs provided herein, an end user will be able to obtain the sequences of probes suitable for HD-PCR, even in silico, and will then be able to generate such probe sequences without the need for amplification. Such probes, whether generated by amplification or by direct synthesis, can be used, inter alia, in interphase and metaphase chromosome spreads.

In general terms, once labeled, the probes are hybridized to a sample containing intact chromosomes, and the binding is analyzed. For example, an interphase or metaphase chromosome preparation may be produced. The chromosomes are attached to a substrate, e.g., glass, contacted with the probe and incubated under hybridization conditions. Wash steps remove all un-hybridized or partially-hybridized probes, and the results are visualized and quantified using a microscope that is capable of exciting the dye and recording images. FISH methods are described in Ried et al., Human Molecular Genetics, Vol 7, 1619-1626; Speicher et al., Nature Genetics, 12, 368-376, 1996; Schrock et al., Science, 494-497, 1996; Griffin et al., Cytogenet Genome Res. 2007; 118(2-4):148-56; Peschka et al., Prenat Diagn., December 1999; 19(12):1143-9; Hilgenfeld et al., Curr Top Microbiol Immunol., 1999, 246: 169-74, the contents of which are incorporated by reference herein.

Prior to in situ hybridization, the FISH probes may be denatured. Denaturation is typically performed by incubating in the presence of high pH, heat (e.g., temperatures from about 70° C. to about 95° C.), organic solvents such as formamide and tetraalkylammonium halides, or combinations thereof.

Intact chromosomes are contacted with labeled probes under in situ hybridization conditions. “In situ hybridization conditions” are conditions that facilitate annealing between a nucleic acid and the complementary nucleic acid in the intact chromosomes. Certain parameters of the hybridization reaction vary, depending on the concentrations, base compositions, complexities, and lengths of the probes, as well as salt concentrations, temperatures, and length of incubation. For example, in situ hybridizations may be performed in hybridization buffer containing 1×-2×SSC, 50% formamide, and blocking DNA to suppress non-specific hybridization. In general, hybridization conditions include temperatures of about 25° C. to about 55° C., and incubation times of about 0.5 hours to about 96 hours. Suitable hybridization conditions for a set of oligonucleotides and chromosomal target can be determined via experimentation which is routine for one of skill in the art. In some embodiments, the hybridization conditions comprise a formamide concentration that may range from about 55-75%, or from about 60-70%, or from about 65-70%, or about 68% formamide. This is above the average formamide concentration of 50% that is typically used in hybridization reactions. It was found in accordance with the invention that shorter probes (e.g., 30-70 bases in length) could perform as well as longer probes (e.g., 100-300 bases) using the same hybridization conditions comprising higher formamide concentrations.

Hybridization to interphase nuclei may be performed using standard protocols including those that employ ethanol dehydration, as well as protocols that employ liquid nitrogen (e.g., 3D FISH protocol developed by Cremer et al. to preserve the 3D architecture of the nucleus).

Fluorescence of a hybridized chromosome can be evaluated using a fluorescent microscope. In general, excitation radiation, from an excitation source having a first wavelength, passes through excitation optics. The excitation optics causes the excitation radiation to excite the sample. In response, fluorescent molecules in the sample emit radiation that has a wavelength that is different from the excitation wavelength. Collection optics then collects the emission from the sample. The computer also can transform the data collected during the assay into another format for presentation. In general, known robotic systems and components can be used.

In certain embodiments, the signal from the binding of the labeled probe to a chromosome may be compared with that of a reference chromosome. The reference chromosome may be from a healthy or wild-type subject. Briefly, the method comprises contacting under in situ hybridization conditions a test chromosome from the cellular sample with a plurality of fluorescently-labeled FISH probes identified and optionally generated by the methods of the invention and contacting under in situ hybridization conditions a reference chromosome with the same plurality of fluorescently-labeled FISH probes. After hybridization, the emission spectra created from the unique binding patterns from the test chromosome are compared against those of the reference chromosome. This and other methods described herein may be carried out using pluralities of chromosomes that may be indistinguishable from each other, such as for example in interphase nuclei.

Thus, the structure of a test chromosome may be determined by comparing the pattern of binding of the labeled FISH probes to the test chromosome with the binding pattern of the same labeled FISH probes with a reference chromosome. The binding pattern of the reference chromosome may be determined before, after or at the same time as the binding pattern for the test chromosome. This determination may be carried out either manually or in an automated system. The binding pattern associated with the test chromosome can be compared to the binding pattern that would be expected for known deletions, insertions, translocation, fragile sites and other more complex rearrangements, and/or refined breakpoints. The matching may be performed by using computer-based analysis software known in the art. Determination of identity may be done manually (e.g., by viewing the data and comparing the signatures by hand), automatically (e.g., by employing data analysis software configured specifically to match optically detectable signature), or using a combination thereof.

In another embodiment, the test sample is from a subject suspected of having a particular condition (e.g., a particular cancer) and the reference sample may comprise a negative control (e.g., non-cancerous) representing a wild-type genome and second test sample (or a positive control) representing the particular condition associated with a known chromosomal rearrangement. In this embodiment, comparison of all these samples with each other may reveal not only if the test sample yields a result that is different from the wild-type genome but also if the test sample may have the same or similar genomic rearrangements as another test (e.g., cancer) sample.

The methods and compositions of the invention find use in myriad of nucleic acid sequence detection applications of interest such as genome mapping, diagnosis, or investigation of various types of genetic abnormalities, cancer or other diseases, including but not limited to, acute and chronic leukemia including chronic myelogenous leukemia (e.g., having translocation between chromosomes 9 and 22) and acute promyelocytic leukemia (e.g., having translocation between chromosomes 15 and 17); lymphoma; multiple myeloma; breast cancer (e.g., having HER2 amplification); lung cancer (e.g., having inversion of chromosome 2 p arm fusing EML4 and ALK genes); colon cancer; prostate cancer; sarcomas and tumors of mesenchymal origin; brain tumors including oligodendroglioma; Alzheimer's disease; Parkinson's disease; epilepsy; amyotrophic lateral sclerosis; multiple sclerosis; stroke; autism; Cri du chat (truncation on the short arm on chromosome 5), 1p36 deletion syndrome (loss of part of the short arm of chromosome 1), Angelman syndrome (loss of part of the long arm of chromosome 15); Prader-Willi syndrome (loss of part of the short arm of chromosome 15); Velocardiofacial syndrome (loss of part of the long arm of chromosome 22); Turner syndrome (single X chromosome); Klinefelter syndrome (an extra X chromosome); Edwards syndrome (trisomy of chromosome 18); Down syndrome (trisomy of chromosome 21); Patau syndrome (trisomy of chromosome 13); and trisomies 8, 9 and 16, which generally do not survive to birth.

The disease may be genetically inherited (germline mutation) or sporadic (somatic mutation). Many exemplary chromosomal rearrangements discussed herein are associated with and are thought to be a factor in producing these disorders. Knowing the type and the location of the chromosomal rearrangement may greatly aid the diagnosis, prognosis, and understanding of various mammalian diseases.

Certain of the above-described methods can also be used to detect diseased cells more easily than standard cytogenetic methods. The above-described methods do not require living cells and can be quantified automatically since a computer can be programmed to count the number and/or arrangement of fluorescent dots present.

The term “nucleic acid” refers to a polymer of any length composed of nucleotides, e.g., deoxyribonucleotides or ribonucleotides. The terms “ribonucleic acid” and “RNA” as used herein mean a polymer composed of ribonucleotides. The terms “deoxyribonucleic acid” and “DNA” as used herein mean a polymer composed of deoxyribonucleotides.

The phrase “labeled probes” refers to nucleic acids that are detectably labeled, e.g., fluorescently labeled, such that the presence of the probe as well as any target sequence to which the probe is bound can be detected by assessing the presence of the label.

The term “hybridization” refers to the specific binding of a nucleic acid to a complementary nucleic acid via Watson-Crick base pairing. The term “in situ hybridization” refers to specific binding of a nucleic acid to a metaphase or interphase chromosome.

The terms “hybridizing” and “binding”, with respect to nucleic acids, are used interchangeably.

The term “stringent assay conditions” refers to conditions that are compatible to produce binding pairs of nucleic acids, e.g., probes and targets, of sufficient complementarity to provide for the desired level of specificity in the assay while being incompatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C. both with a wash of 0.2×SSC and 0.1% SDS at 65° C. Other stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions determines whether a nucleic acid is specifically hybridized to a probe. Wash conditions used to identify nucleic acids may include, e.g. a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen for detailed descriptions of equivalent hybridization and wash conditions and for reagents and buffers, e.g., SSC buffers and equivalent reagents and conditions.

In some embodiments, a 3D-FISH buffer is used that comprises 68% formamide, 1.7×SSC, 10% dextran sulphate, 50 mM Na2HPO4/NaH2PO4, 1 mM EDTA, and 5×Denhardt's solution.

In some embodiments, a universal buffer is used (e.g., for all other fixation procedures including for example methanol-acetic acid fixation). This universal buffer comprises 50% formamide, 1.7×SSC, 10% dextran sulphate, 50 mM Na2HPO4/NaH2PO4, 1 mM EDTA, and 5×Denhardt's solution.

The terms “plurality”, “set” or “population” are used interchangeably to mean 2 or more, including up to 5, 10, 20, 30, 40, 50, 60, 70, 80, 90, 100, 500, 1000, 10000, 100000, 1000000, 10,000,000, or 100,000,000.

The term “chromosomal region” refers to a contiguous length of nucleotides in a genome of an organism. A chromosomal region may be in the range of 10 kb in length to an entire chromosome, e.g., 100 kb to 10 MB for example.

The term “banding pattern” refers to the pattern of banding of a set of labeled probes to an intact chromosome.

The term “genome” refers to all nucleic acid sequences (coding and non-coding) and elements present in or originating from any prokaryotic or eukaryotic organism. The term genome also applies to any naturally occurring or induced variation of these sequences that may be present in a mutant or disease variant of any cell type. For example, the human genome consists of approximately 3×10⁹ base pairs of DNA organized into distinct chromosomes. The genome of a normal diploid somatic human cell consists of 22 pairs of autosomes (chromosomes 1 to 22) and either chromosomes X and Y (males) or a pair of chromosome X (female) for a total of 46 chromosomes. A genome of a cancer cell may contain variable numbers of each chromosome in addition to deletions, rearrangements and amplification of any sub-chromosomal region or DNA sequence.

The term “genomic sample” refers to a material or mixture of materials, containing genetic material from a species. The term “genomic DNA” as used herein refers to deoxyribonucleic acids that are obtained from a species. The terms “genomic sample” and “genomic DNA” encompass genetic material that may have undergone purification, fragmentation, or amplification. The genomic sample may be prepared using any convenient protocol. In many embodiments, the genomic sample is prepared by first obtaining a starting composition of genomic DNA, e.g., a nuclear fraction of a cell lysate, where any convenient means for obtaining such a fraction may be employed and numerous protocols for doing so are well known in the art. In certain embodiments, the genomic sample may comprise a portion of the genome, e.g., one or more specific chromosomes or regions thereof, such as PCR amplified regions produced with a pairs of specific primers.

The term “chromosomal rearrangement” refers to an event where one or more parts of a chromosome are rearranged within a single chromosome or between chromosomes. In certain cases, a chromosomal rearrangement may reflect an abnormality in chromosome structure. A chromosomal rearrangement may be an inversion, a deletion, an insertion or a translocation, for example.

The term “primer” refers to an oligonucleotide that has a nucleotide sequence that is complementary to a region of a nucleic acid to be amplified. A primer binds to the complementary region and is extended, using the target nucleic acid as the template, under primer extension conditions. A primer may be in the range of about 5 to about 20 nucleotides although primers outside of this length are envisioned.

The term “genomic distance” means the number of nucleotides separating two probe positions on the contiguous sequence of interest. The probe position may be determined in terms of the 5′ most nucleotide of a probe and the genomic distance is the number of nucleotide bases separating the 5′ most nucleotide of the two probes.

As stated above, the methods of the invention may be performed using a computer system. An example of a computer system that may be used in connection with any of the embodiments of the invention described herein may include one or more processors and one or more computer-readable non-transitory storage media (e.g., memory and one or more non-volatile storage media). The processor may control writing data to and reading data from the memory and the non-volatile storage device in any suitable manner, as the aspects of the present invention described herein are not limited in this respect. To perform any of the functionality described herein, the processor may execute one or more instructions stored in one or more computer-readable storage media (e.g., the memory), which may serve as non-transitory computer-readable storage media storing instructions for execution by the processor.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation comprises at least one non-transitory computer-readable storage medium (e.g., a computer memory, a floppy disk, a compact disk, a tape, etc.) encoded with a computer program (i.e., a plurality of instructions), which, when executed on a processor, performs the above-discussed functions of the embodiments of the present invention. The computer-readable storage medium can be transportable such that the program stored thereon can be loaded onto any computer resource to implement the aspects of the present invention discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

The following Examples are included for purposes of illustration and are not intended to limit the scope of the invention.

### EXAMPLES

### Example 1

### Materials

### Cells and Tissues

hTERT-HME1 mammary epithelium cells were kindly provided by R. Weinberg (MIT) and grown in the MEGM medium from LONZA (#CC-3151). Metaphase spreads derived from human lymphocytes were obtained from Abbott Diagnostics (#30-806010). For HER2 RNA-DNA FISH in breast cancer, HER2 IHC control tissue arrays with ten cores mounted on #1 coverglasses coated with poly-L-lysine were purchased from US Biomax. After deparaffinization in D-Limonene (VWR), tissue sections were re-hydrated, post-fixed 10 min in 4% paraformaldehyde in 1×PBS solution, heated 45 min at 80° C. in 0.01M sodium citrate pH 6, and then treated with 0.025% pepsin (Sigma) in 0.01M HCl. Auto-fluorescence was reduced by immersing tissue sections in NaBH₄ 1% in 1×PBS solution for 10 min at room temperature. After three washes 10 min each in RNAse-free water, samples were stored in 2×SSC buffer supplemented with Ribonucleoside Vanadyl Complex (see below) at 4° C. until hybridization was performed.

### PCR Reagents

Primers for PCR were purchased from Integrated DNA Technologies (IDT) in 96-well plates at 100 μM concentration in RNAse-free water. KAPA SYBR FAST qPCR Master Mix 2× was from KAPABiosystems. Human male genomic DNA (Promega #G1471) was used as template.

### FISH Reagents

Reagents for FISH were purchased from the following companies. Formamide (#AM9342), 20×SSC (#AM9765), 1M Tris, 10×PBS, 5% BSA (#AM2616), RNAseZAP (#AM9780M), RNAsin (#N2615), and SUPERasin (#AM2696) were from Ambion. Protector was from ROCHE (#03335399001). 100×Denhardt's solution was obtained from Amresco (#E257). The following reagents were obtained from Sigma: Catalase (#3515), Glucose Oxidase (#G2133), Trolox (#238813), Igepal (#CA-630), Dextran sulfate (#D8906), E. coli tRNA (#R4251), and 20 mg/mL Glycogen (#G1767). Tween20 and Triton X-100 were from Promega (#H5151 and #H5141, respectively). RNAseA 100 mg/mL was from Qiagen. 1N HCl was from EMS (#16770). Fixogum was from Kreatech (#LK-071). Ribonucleoside Vanadyl Complex (RVC) was from NEB (# S1402S). Human Cot-1 DNA was from Invitrogen (#15279-011).

HD-FISH hybridization buffer for metaphase spreads contained 1.7×SSC, 50% formamide, 50 mM Na2HPO4/NaH2PO4, 10% dextran sulfate, and 5×Denhardt's solution pH 7.5.

HD-FISH hybridization buffer for interphase cells contained 1.7×SSC, 70% formamide, 50 mM Na2HPO4/NaH2PO4, 10% dextran sulfate, and 5×Denhardt's solution pH 7.5.

RNA FISH hybridization buffer consisted of 25% formamide, 2×SSC, 10% dextran sulfate, 1 mg/ml E. coli tRNA, 0.2% BSA, and 20 mM RVC.

The HER2 RNA FISH probe consisted of 48 different 20nt-long oligonucleotide sequences, each having the 3′ covalently bound to an amino group and purchased from Biosearch Technologies. The probe was coupled to AlexaFluor594 (Invitrogen) as previously described.

For microscopy, samples were covered with a mounting solution containing 2×SSC buffer, 10 mM Tris, 0.4% glucose, Catalase, 37 μg/mL Glucose Oxidase, and 2 mM Trolox.

### Methods

### Synthesis of HD-FISH Probes by PCR

For each probe, forward and reverse primers were synthesized in corresponding wells of separate 96-well plates (stock plates). Forward and reverse primer pairs were mixed and diluted at 5 μM in TE buffer pH 8 using clear real-time PCR plates (clear LightCycler 480 Multiwell Plate 96, Roche) (dilution plates) at well positions matching the position of primers in the stock plate. For each probe, real-time PCR reactions were carried out in the same type of plates (experimental plates), by transferring the appropriate volume of 5 μM primer dilution from the dilution plate to the corresponding well in the experimental plate. For each reaction (in one well), the following volumes were used:

| REAGENT | VOLUME (μL) |
|---|---|
| KAPA SYBR FAST qPCR Master Mix 2x | 25 |
| 5 μM Forward + Reverse primers dilution | 4 |
| Human male genomic DNA (diluted at 100 ng/μL in TE | 1 |
| buffer pH 8) | |
| Nuclease-free water | 20 |

For each plate, real-time PCR reactions were performed in a LightCycler® 480 instrument (Roche) according to the following program:

| CYCLE | STEP | TEMP. | TIME | ACQUISITION MODE |
|---|---|---|---|---|
| Pre-amplification | 1 | 95° C. | 5 min | None |
| Amplification | 1 | 95° C. | 10 sec | None |
| | 2 | 55° C. | 10 sec | None |
| | 3 | 72° C. | 10 sec | Single |
| Cooling | 1 | 40° C. | 10 sec | None |

Amplification through steps 1-3 was repeated 30 times. After PCR, the contents of all wells corresponding to a given probe were pooled together in a sterile cell culture basin (VWR), and aliquoted into 1.5 mL tubes for subsequent ethanol precipitation. Wells in which either no product was observed or in which the amplification kinetics was significantly different than in all other wells were excluded. Ethanol precipitation was carried out by adding 3M sodium acetate pH 5.5 to PCR aliquots at 10% vol./vol. ratio, followed by addition of 2.5 volumes of 100% ice-cold ethanol and incubation overnight at −20° C. The next day, DNA pellets were washed twice in 70% ice-cold ethanol, air-dried for 20 min, and re-dissolved in nuclease-free water. DNA concentration was measured using Nanodrop.

### HD-FISH Probes Labeling

For each probe, a volume corresponding to 1 μg was lyophilized in a 1.5 mL tube, and then labeled with the fluorophore of interest using the corresponding ULYSIS Nucleic Acid Labeling Kit (Invitrogen) according to the manufacturer's instructions. Unbound dyes were removed by gel filtration using one KREApure column per probe following the manufacturer's instructions (Kreatech). Labeled probes were stored at −20° C., and were stable at this temperature up to six months.

### Preparation of HD-FISH Hybridization Solution

200 ng (in case of HER2 probes) or 20 ng (in case of spotting probes simultaneously targeting 10 different loci on chromosome 17) of probe were precipitated using the following protocol:

| | | | Nuclease- | | Ethanol |
|---|---|---|---|---|---|
| PROBE | Cot1 DNA | Glycogen | free water | Na-acetate | 100% |
| 200/20 ng | 2.5 μg | 20 μg | Up to 100 μL | 11 μL | 300 μL |

The mix was incubated 2 h at −20° C., then DNA pellets were washed twice in 70% ice-cold ethanol, air-dried for 20 min, and re-dissolved in 20 μL (in case of cells) or 30 μL (in case of breast cancer tissue) of the appropriate hybridization buffer. The hybridization solution was incubated at 55° C. for 1 h while moderately shaking to facilitate solubilization of DNA.

HD-FISH on Metaphase Spreads Metaphase spread slides were denatured for 5 min at 75° C. in a solution containing 70% formamide and 2×SSC pH 7. Afterwards, the slides were immersed in 70% ethanol at room temperature for 2 min, followed by the same treatment in 80% and 100% ethanol. The slides were then air-dried for 20 min. Meanwhile, the desired probe was denatured at 75° C. for 5 min, after which it was applied on dry slides and sealed with Fixogum. The slides were then incubated at 37° C. for 24 h. After hybridization Fixogum was removed and the slides were washed first in 0.4×SSC with 0.3% Igepal at 73° C. for 2 min, and then in 2×SSC with 0.1% Igepal for 1 min at room temperature. Finally slides were transferred into 2×SSC buffer supplemented with 100 ng/mL DAPI, and incubated for 5 min at room temperature. Afterwards the slides were rinsed in 2×SSC and covered with mounting solution before imaging.

### 3D HD-FISH on Interphase Nuclei

hTERT-HME1 cells were plated at a density of 10⁶ cells per one 10 cm culture dish in MEGM medium. Every dish contained 5 22×22 mm sterilized #1 coverglasses coated with 0.2% gelatin. Two days after plating, when cells reached a confluency of approx. 90-100%, they were processed as follows: dishes were rinsed with 1×PBS and incubated with 0.3×PBS for 1 min at room temperature. Afterwards cells were fixed in 4% formalin in 0.3×PBS for 10 min at room temperature, washed 3 times with 1×PBS containing 0.05% Triton X-100, and permeabilized with 1×PBS containing 0.5% Triton X-100 for 20 min at room temperature. After that, cells were rinsed with 20% glycerol in 1×PBS and incubated in 20% glycerol in 1×PBS overnight. The next day all coverglasses were individually subjected to 6 freeze-thaw cycles in liquid nitrogen (25 sec in liquid nitrogen, followed by gradual de-frosting, followed by approx. 2 min in glycerol solution before the next cycle). Coverglasses were washed 3 times with 1×PBS containing 0.05% Triton X-100, and then incubated in 0.1N HCl solution for 5 min at room temperature. Afterwards coverglasses were washes twice with 1×PBS containing 0.05% Triton X-100, once with 2×SSC, then rinsed in 2×SSC buffer with 50% formamide pH 7.0, and finally incubated in the same solution overnight. After this preparation, coverglasses were stored at 4° C. for 3 days up to two months and used for hybridization whenever needed.

Coverglasses stored in 2×SSC buffer with 50% formamide pH 7 at 4° C. were gradually allowed to reach room temperature before applying a probe onto them. 20 μL of the desired hybridization solution were applied onto a microscope slide, covered with the coverglass coated with cells, and sealed with Fixogum. After Fixogum had solidified, slides were hybridized for 3-5 min at 75° C. (coverglasses that had been stored in 2×SSC 50% formamide for less than a month were denatured for 5 min, whereas those kept longer were denatured for 3 min). Hybridization was allowed to proceed at 37° C. for 24 h in a humidity chamber.

After hybridization coverglasses were removed from slides and washed as follows: they were rinsed in 2×SSC and washed twice in 0.2×SSC with 0.2% Tween20 at 56° C. for 7 min each. Then they were rinsed with 4×SSC containing 0.1% Tween20, and incubated in 2×SSC containing 100 ng/mL DAPI for 5 min at room temperature. Samples were covered with mounting solution before imaging.

3D HD-FISH Combined with RNA FISH on Interphase Cells

All steps until permeabilization (included) were the same as for 3D HD-FISH only (see above). Starting from fixation on, exceptional care was taken in order to avoid RNAse contamination. Hence, all solutions were prepared using RNAse-free water generated by a Barnstead water purification system equipped with the UF filter (ThermoFisher Scientific, #D8611). Whenever available, certified RNAse-free reagents were used. If this was not possible, the RNAse inhibitor RVC was added to a 20 mM final concentration. The Dewar flask in which liquid nitrogen freeze/thaw cycles were performed as well as all the tweezers and microscope slides used were rendered RNAse-free by RNAseZAP treatment prior to use.

After permeabilization cells were rinsed with 20% glycerol in 1×PBS and incubated in 20% glycerol in 1×PBS for 3 h at room temperature, and then at 4° C. overnight. The next day all coverglasses were subjected to 6 freeze-thaw cycles in liquid nitrogen and processed for HD-FISH as described above.

After HD-FISH hybridization, microscope slides were immersed in 2×SSC solution supplemented with 20 mM RVC to facilitate Fixogum removal. After being released, coverglasses were transferred onto 100 μL of HER2 RNA FISH hybridization solution dispensed onto a piece of Parafilm, and incubated at 30° C. for 3 h. Afterwards coverglasses were washed twice at 30° C. for 30 min in 25% RNA WASH buffer composed of 2×SSC and 25% formamide (the second wash included 20 ng/mL DAPI). After washes, coverglasses were rinsed with 2×SSC and covered with mounting solution before imaging.

HD-FISH Combined with RNA FISH on Breast Cancer Tissue Arrays

Coverglasses coated with breast cancer tissue arrays in 2×SSC solution with 20 mM RVC stored at 4° C. (see above) were allowed to gradually reach room temperature, and then were transferred into 2×SSC with 50% formamide pH 7, and incubated overnight at room temperature. The next day coverglasses were placed onto 30 μL of hybridization solution containing 200 ng of 50 kb HER2 probe together with 1 μL of Protector solution. Coverglasses were sealed with Fixogum and placed at 37° C. for 3 h before being denatured in order for the probe to fully penetrate the tissue. Samples were then denatured at 85° C. for 5 min and hybridization was carried on for 40 h at 37° C. in a humidity chamber. All steps afterwards were identical to those for 3D HD-FISH combined with RNA FISH on interphase cells (see above).

### Results

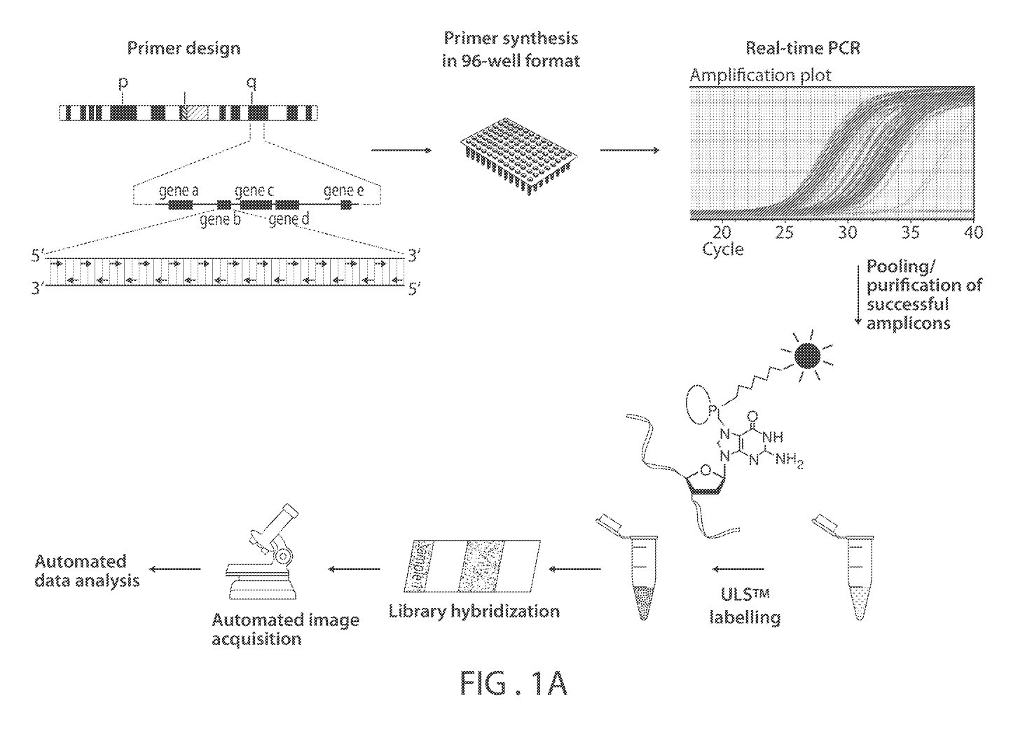

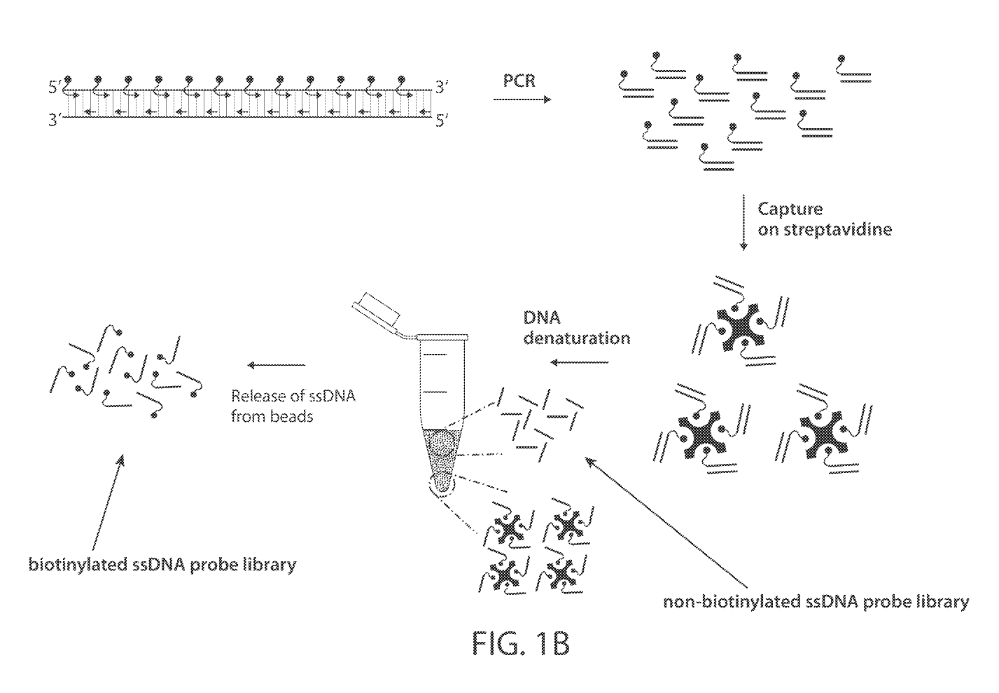

We have developed a high-definition DNA Fluorescent In Situ Hybridization (HD-FISH) method, which is based on rapid and cost-effective PCR-based synthesis of genome-wide probe libraries. Using a custom-made algorithm, we have compiled a list of PCR primer pairs with optimal sequence and thermodynamic properties along the entire human genome. For a given region of interest, a set of 10-60 such primer pairs is selected, synthesized in 96-well plates, and used to generate ˜200 bp-long unique DNA fragments by real-time PCR. These fragments are subsequently pooled, purified, and may be fluorescently labeled en mass using for example the Universal Linkage System chemistry (ULS™, Kreatech), thus finally serving as a ready-to-use HD-FISH probe (FIG. 1A). Optionally, either forward or reverse primers can be synthesized with a 5′-biotin group, allowing the synthesis of strand-specific, single-stranded probes (FIG. 1B).

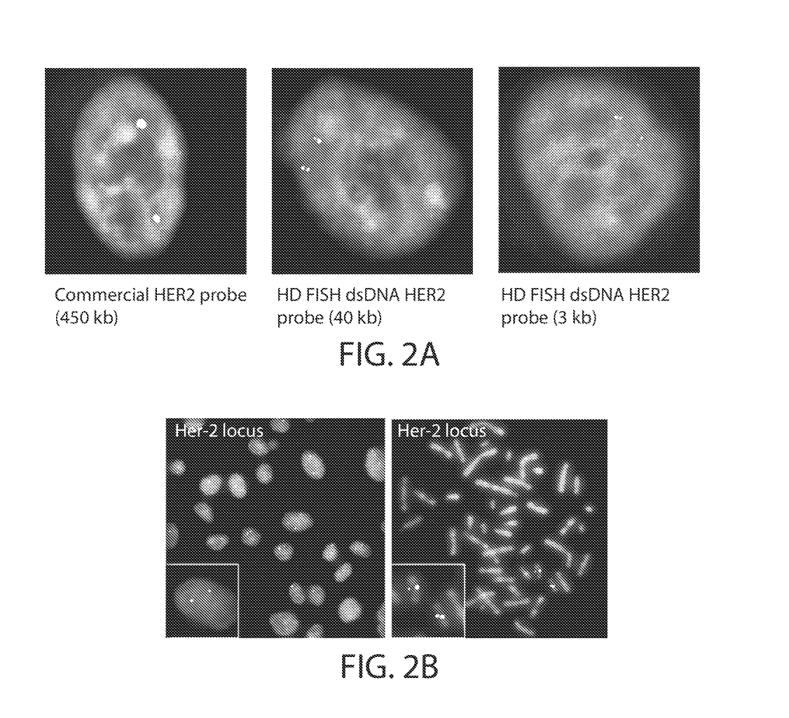

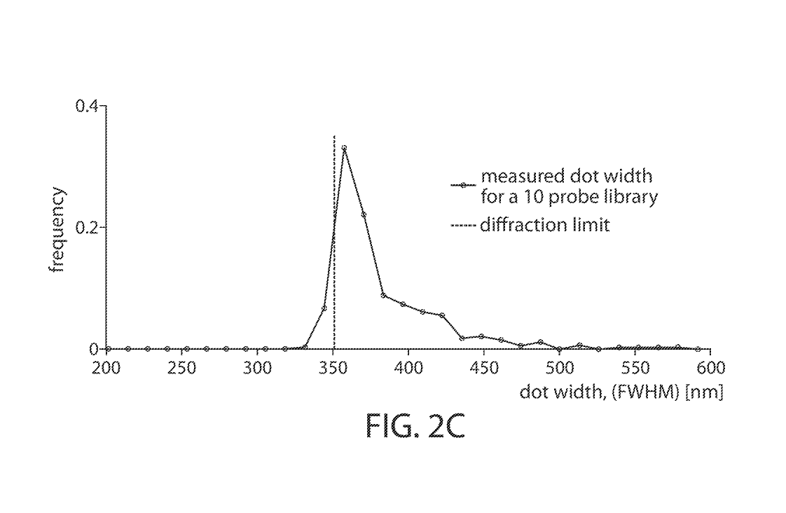

In pilot experiments, we have tested our probe design and synthesis method by constructing probes targeting the 40 kb ERBB2/HER2 locus on human chromosome 17. This locus is amplified in 25-30% of all ductal invasive breast adenocarcinomas, and DNA FISH is the recommended method to assess the HER2 copy number in tumors scored 1-2+ in immunohistochemistry⁶. While a commercially available probe typically yields patchy signals difficult to automatically identify, both single- and double-stranded probes prepared with our method produce bright, punctuated signals that can be easily identified and automatically counted in interphase nuclei (FIG. 2A). The positions of these signals can also be precisely determined, in contrast to the patchy signals observed with traditional DNA FISH probes. Notably, using a probe consisting of only ten 200 bp-long fragments targeting a region as short as 3 kb, a diffraction-limited fluorescent signal can be obtained (FIG. 2B). Diffraction-limited fluorescent signals appear as small (˜350 nm in diameter), discrete spots by high-resolution bright-field or confocal microscopy. These signals are optimally suited for automatic image processing as they can be described with relatively simple mathematical functions. The probes used for the 3 kb experiments were generated using the HER2 3 kb primer set provided herein.

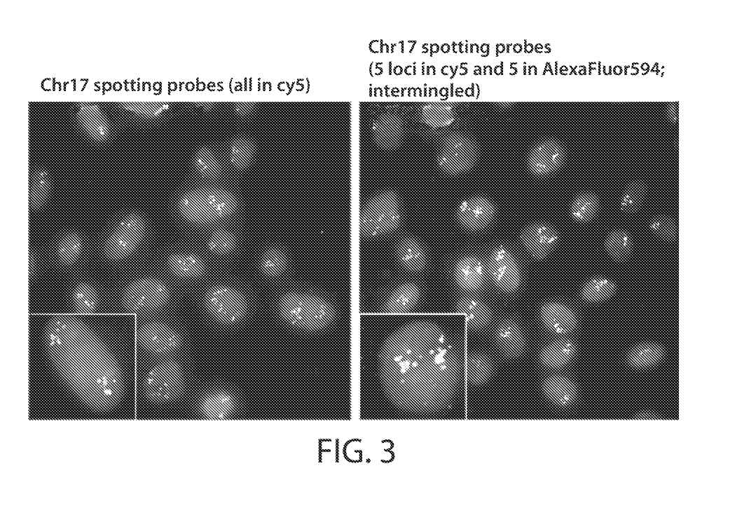

We have also analyzed whether the methods set forth herein can be used to prepare chromosome paint probes. Chromosome paint probes are specific for either entire chromosomes or parts thereof (e.g., the p or q arm). Such probes are difficult to produce and expensive to purchase. These probes hold important biomedical applications, such as in the diagnosis of pathologic chromosome number aberrations (e.g. X monosomy in Turner's syndrome or 21 trisomy in Down's syndrome). In principle, using our method, a set of chromosome-specific “spotting probes” can be rapidly synthesized, and used to visualize a chromosome territory in interphase nuclei as a “cloud” of dots. To test the feasibility of this idea, we generated probes targeting ten genes on chromosome 17, separated by approximately 8 Mb and labeled with the same fluorophore. After mixing all the probes and using the pool for HD-FISH, computationally identifiable spot clusters, each marking one chromosomal territory, could be detected (FIG. 3).

In conclusion, compared to traditional DNA FISH which is based on laborious and time-consuming preparation of probes from bacterial artificial chromosomes (BACs) or laser-captured chromosome fragments, our method allows for streamlined and cost-effective preparation of probes. The preparation of a chromosome spotting probe useful for over 100 tests, and consisting of a mix of 10 different gene-targeting probes, requires 1.5 working days and less than 2000 $ for primer synthesis. Moreover, unlike traditional FISH, HD-FISH of the invention yields signals amenable to robust and automated computational processing in a large number of nuclei. Lastly, our method allows systematic visualization of regions as small as 3 kb together with discrimination between the Watson and Crick strands of the double helix. Thus, we envision that our HD-FISH method will have a broad range of applications, including medical diagnostics, single-cell in situ karyotyping, chromatin architecture studies, and analysis of combed DNA.

### Example 2

A comparison of the method of the invention to a method reported by Navin N., et. al. 2006. Bioinformatics (referred to as the “Prober” method) was performed. In the comparison, primer sets for 10 randomly-selected 100 kb regions of chromosome 17 generated using Prober and the methods of the invention were compared.

The Prober software was used to design primer pairs with relaxed constraints, relative to the default settings and compared to the examples that accompanied the Navin et al. published reference. The following settings were relaxed (1) mer.count.cutoff=2 (default=1), (2) Max Repeats=5 (default=4), (3) Minimum Melting Temperature=55 (instead of 60), and (4) Maximum Melting Temperature=80 (instead of 75). The final Prober probe lengths were constrained to between 200-1,000 bp. This is significantly higher than the relatively uniform length distribution imposed upon the probe set of the invention which in this examples was 200-220 bp.

According to the Navin et al. published reference, the final probe set must cover at least 20% of the target sequence. However, none of the 10 regions tested produced Prober sets with enough probes to satisfy the minimum quality cutoff. The total number of probes ranged from 0 to 37, with the total coverage between 0-18%. In contrast, using the method of the invention, all ten regions had numerous 200 bp probes. Our totals ranged between 169 and 251 probes, with coverage of the target regions of about 35-53%. Table 3 provides the raw data of this comparative study.

TABLE 3

| | | Percent | | |
|---|---|---|---|---|
| | Total Probes | Coverage | Total Probes | Percent Coverage |
| Location | (PROBER) | (PROBER) | (Invention) | (Invention) |
| 5 mb | 22 | 10.9 | 239 | 49.8 |
| 14 mb | 0 | 0 | 183 | 38.2 |
| 21 mb | 31 | 13.6 | 192 | 40.1 |
| 29 mb | 17 | 7.4 | 171 | 35.6 |
| 37 mb | 15 | 6.2 | 206 | 43 |
| 46 mb | 8 | 3.6 | 185 | 38.6 |
| 53 mb | 3 | 1.7 | 226 | 47.1 |
| 62 mb | 15 | 7 | 194 | 40.1 |
| 70 mb | 9 | 3.5 | 251 | 52.5 |
| 78 mb | 37 | 17.5 | 169 | 35.2 |

Even more significantly, requiring a uniform probe size of 200-300 bp (still broader than our imposed length range) results in complete failure of the Prober software to generate any probes.

Accordingly, the Prober software and method is not able to generate, inter alia, a set of FISH PCR-based probes that are under 1 kb in length, including probes ranging in length from about 70 bp to about 300 bp and uniform probes in that range (e.g., about 200 base pairs) that can be applied to genomic DNA regions of 10 kb or more.

### REFERENCES

1. South, S. T., Chen, Z. & Brothman, A. R. Genomic medicine in prenatal diagnosis. Clin Obstet Gynecol 51, 62-73 (2008).
2. Sreekantaiah, C. FISH panels for hematologic malignancies. Cytogenet Genome Res 118, 284-96 (2007).
3. Cremer, T. & Cremer, M. Chromosome territories. Cold Spring Harb Perspect Biol 2, a003889.
4. Hubner, M. R. & Spector, D. L. Chromatin dynamics. Annu Rev Biophys 39, 471-89.
5. Takizawa, T., Meaburn, K. J. & Misteli, T. The meaning of gene positioning. Cell 135, 9-13 (2008).
6. Clinical laboratory assays for HER-2/neu amplification and overexpression: quality assurance, standardization, and proficiency testing. Arch Pathol Lab Med 126, 803-8 (2002).
7. Raj, A., van den Bogaard, P., Rifkin, S. A., van Oudenaarden, A. & Tyagi, S. Imaging individual mRNA molecules using multiple singly labeled probes. Nat Methods 5, 877-9 (2008).

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The patent application contains at least one drawing executed in color. Copies of this patent application publication with color drawing(s) will be provided by the Office upon request and payment of the necessary fee.

1. A method comprising
(a) identifying a plurality of nucleic acids, each having a nucleotide sequence that is unique relative to known sequence of genomic DNA of a species,
(b) identifying, from each unique nucleic acid identified in (a), 30-300 bp nucleic acid fragments and amplification primer pairs that yield 70-300 base pair nucleic acid fragments, thereby generating a putative probe set and a putative primer set,
(c) eliminating, from the putative probe set, nucleic acids having amplification primer pairs that amplify more than one nucleic acid fragment in genomic DNA of the species, and optionally eliminating, from the putative primer set, amplification primer pairs that amplify more than one nucleic acid fragment in genomic DNA of the species, and
(d) eliminating, from the putative probe set, nucleic acid fragments that hybridize to other regions in the genomic DNA of the species, and optionally eliminating, from the putative primer set, amplification primer pairs that amplify such nucleic acid fragments,
thereby producing an HD-FISH probe set and/or an HD-FISH primer set.

2. The method of claim 1, wherein step (a) is performed by analyzing a plurality of overlapping nucleic acid sequences along a chromosome.

3. The method of claim 2, wherein each of the overlapping nucleic acid sequences is about 500 bases in length.

4. The method of any one of the foregoing claims, wherein the species is human.

5. The method of any one of the foregoing claims, wherein step (a) is performed in silico.

6. The method of any one of the foregoing claims, wherein step (c) is performed in silico.

7. The method of any one of the foregoing claims, wherein step (d) is performed in silico.

8. The method of any one of the foregoing claims, wherein step (d) is more stringent than step (a).

9. The method of any one of the foregoing claims, wherein the HD-FISH probe set comprises one or more subsets of 50 or more probes that bind to single 100 kb regions of sequenced human genomic DNA.

10. The method of any one of the foregoing claims, wherein the HD-FISH probe set comprises multiple subsets of 50 or more probes that bind to 93% of 100 kb regions of sequenced human genomic DNA.

11. The method of any one of the foregoing claims, wherein the HD-FISH probe set comprises probes that are about 40-300 bases in length.

12. The method of any one of the foregoing claims wherein step (c) is performed before step (d).

13. The method of any one of the foregoing claims wherein step (d) is performed before step (c).

14. The method of any one of the foregoing claims, further comprising synthesizing one or more subsets of probes within the HD-FISH probe set using an amplification reaction.

15. The method of claim 14, wherein the amplification reaction is a PCR reaction.

16. The method of any one of claims 1-13, further comprising synthesizing one or more subsets of probes directly.

17. A composition comprising
an HD-FISH probe set that comprises multiple probe subsets each comprising 50 or more probes that in total hybridize to 93% of discrete 100 kb regions of sequenced human genomic DNA.

18. The composition of claim 17, wherein the HD-FISH probe set comprises probes that are about 30-300 bases in length, about 40-300 bases in length, or about 70-300 bases in length.

19. The composition of claim 17 or 18, wherein probes within the HD-FISH probe set are fluorescently labeled.

20. The composition of claim 17, 18 or 19, wherein the HD-FISH probe set is produced by performing a polymerase chain reaction on genomic DNA in the presence of an HD-FISH primer set.

21. The composition of claim 20, wherein probes within the HD-FISH probe set are uniformly fluorescently labeled.

22. The composition of claim 19, 20 or 21, wherein probes within the HD-FISH probe set are fluorescently 5′ end-labeled.

23. The composition of any one of claims 17-22, wherein the probes are single-stranded.

24. A composition comprising an HD-FISH probe set produced according to the method of any one of claims 1-16.

25. A composition comprising
an HD-FISH primer set that amplifies an HD-FISH probe set comprising one or more primer pair subsets each having 50 or more probes that together hybridize to 93% of discrete 100 kb regions of sequenced human genomic DNA.

26. A composition comprising an HD-FISH primer set identified according to the method of any one of claims 1-13 and produced using an enzyme-dependent or an enzyme-independent process.

27. The composition of claim 25 or 26, wherein probes within the HD-FISH probe set are fluorescently labeled.

28. The composition of claim 27, wherein probes within the HD-FISH probe set are uniformly fluorescently labeled.

29. A method comprising
performing a fluorescent in situ hybridization (FISH) reaction in the presence of an HD-FISH probe set, or a subset thereof, that hybridizes to a 10 kb region of interest, wherein the probes are about 30 to about 300 bases in length, and wherein a FISH result that differs from a control indicates a chromosomal abnormality, wherein the reaction is performed in more than 50% formamide (v/v).

30. The method of claim 29, wherein the reaction is performed in more than 60% formamide (v/v).

31. The method of claim 29, wherein the reaction is performed in more than 70% formamide (v/v).

32. The method of claim 29, 30 or 31, wherein the probes are about 40-300 bases in length.

33. The method of claim 29, 30 or 31, wherein the probes are about 40, about 50, about 60, about 70, about 80, about 90, or about 100 bases in length.

34. A method comprising
performing a fluorescent in situ hybridization (FISH) reaction in the presence of an HD-FISH probe set, or a subset thereof, that hybridizes to a 3 kb region of interest, wherein the probes are about 70 to about 300 bases in length, and wherein a FISH result that differs from a control indicates a chromosomal abnormality.

35. The method of any one of claims 29-34, wherein the HD-FISH probe set or subset thereof comprises 5-30 probes.

36. The method of any one of claims 29-34, wherein the HD-FISH probe set or subset thereof comprises 10 probes.

37. The method of any one of claims 29-36, further comprising synthesizing the HD-FISH probe set or a subset thereof using an amplification method.

38. The method of claim 37, wherein the amplification method is a PCR amplification.

39. The method of claim any one of claims 29-36, further comprising directly synthesizing the HD-FISH probe set or a subset thereof.
